(19) United States
(12) Patent Application Publication
(10) Pub. No.: US 2011/0038807 A1
(43) Pub. Date: Feb. 17, 2011

(54) COMPOSITIONS AND METHODS FOR TREATING BIPOLAR DISORDER

(76) Inventor: Demitri Papolos, Westport, CT (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/541,403

(22) Filed: Aug. 14, 2009

(51) Int. Cl. A61K 9/12; A61K 31/135

(52) U.S. Cl. 424/45; 514/647; 424/46

(57) ABSTRACT

The present invention is directed to a method for treating bipolar disorder, childhood-onset bipolar disorder, and associated symptoms. The method includes administering to a subject in need of such treatment a dose of ketamine sufficient to alleviate symptoms associated with bipolar disorder and childhood-onset bipolar disorder.

### FIELD OF THE DISCLOSURE

This application describes therapeutic compositions comprising ketamine. The compositions may be used in methods for treating bipolar disorder.

### BACKGROUND OF THE DISCLOSURE

Bipolar disorder, also known as manic depression, manic depressive disorder or bipolar affective disorder, is a psychiatric diagnosis that describes a category of mood disorders defined by the presence of one or more episodes of abnormally elevated mood clinically referred to as mania or, if milder, hypomania.

People with bipolar disorder experience unusually intense emotional states that occur in distinct periods called “mood episodes.” An overly joyful or overexcited state is called a manic episode, and an extremely sad or hopeless state is called a depressive episode. Individuals who experience manic episodes also commonly experience depressive episodes or symptoms, or mixed episodes in which features of both mania and depression are present at the same time. These episodes are usually separated by periods of “normal” mood, but in some individuals, depression and mania may rapidly alternate, known as rapid cycling. Extreme manic episodes can sometimes lead to psychotic symptoms such as delusions and hallucinations. The disorder has been subdivided into bipolar I, bipolar II, cyclothymia, and other types, based on the nature and severity of mood episodes experienced; the range is often described as the bipolar spectrum.

Patients affected by bipolar disorder have had at least one manic or hypomanic (mild mania) episode. However, at the time of diagnosis, they may never have had a depressive episode, according to the DSM-IV criteria. The diagnosis is supported by family history data and observational studies. According to the DSM-IV, patients with full manias and depression are indicated as having “bipolar I disorder,” while patients with hypomanias and depressions are described as having “bipolar II disorder.” Onset of episodes tends to be acute, with symptoms developing over days to weeks. The depressive episodes of bipolar patients are indistinguishable from those of patients with unipolar disorder. Thus, misdiagnosis of bipolar disorder is common. Indeed, as many as 40 percent of bipolar patients are initially misdiagnosed.

Symptoms of mania or a manic episode include both mood changes and behavioral changes. Mood changes include the following: a long period of feeling “high,” or an overly happy or outgoing mood; and extremely irritable mood, agitation, feeling “jumpy” or “wired.” Behavioral Changes include the following: talking very fast, jumping from one idea to another, having racing thoughts; being easily distracted; increasing goal-directed activities, such as taking on new projects; being restless; sleeping little; having an unrealistic belief in one's abilities; behaving impulsively and taking part in a lot of pleasurable; and high-risk behaviors, such as spending sprees, impulsive sex, and impulsive business investments.

Symptoms of depression or a depressive episode include both mood changes and behavioral changes. Mood changes include the following: a long period of feeling worried or empty; and loss of interest in activities once enjoyed, including sex. Behavioral Changes include the following: feeling tired or “slowed down”; having problems concentrating, remembering, and making decisions; being restless or irritable; changing eating, sleeping, or other habits; and thinking of death or suicide, or attempting suicide.

It was traditionally believed that bipolar disorder could, at the earliest, develop in a person's late teens or early adult. Indeed, many parents are told that the diagnosis cannot be made until the child grows into the upper edges of adolescence—between 16 and 19 years old. Bipolar disorder, however, also affects close to 1 million children and adolescents in the United States at any given time. A proper diagnosis of childhood-onset (or early-onset) bipolar disorder may be made in a child as early as early as 18 months.

Bipolar disorder manifest differently in children or adolescents as compared to adults. Adults experience abnormally intense moods for weeks or months at a time, but children appear to experience rapid shifts of mood that they commonly cycle many times within the day. This cycling pattern associated with low arousal states in the mornings followed by afternoons and evenings of increased energy. Nonetheless, the Diagnostic and Statistical Manual of Psychiatry—the DSM-IV—uses the same criteria to diagnose bipolar disorder in children as it does to diagnose the condition in adults, and requires that the manic and depressive episodes last a certain number of days or weeks. Thus, bipolar children experiencing a much more chronic, irritable course, with many shifts of mood in a day, will not meet the duration criteria of the DSM-IV.

Treatment of bipolar disorder can be problematic. In adults, mania requires prompt treatment because it can rapidly worsen, resulting in poor judgment that endangers interpersonal relationships, jobs, and finances. Management is founded upon medication, provision of a low-stimulation environment, and protecting the patient from undertaking potentially harmful activities. Initial management of acute mania is often best accomplished through hospitalization. Thus, the management of bipolar disorder can be expensive, intrusive, and difficult. In addition, despite the now routine use of maintenance treatment for bipolar disorder, up to 90 percent of patients experience at least one relapse within 5 years of their original diagnosis.

Adult bipolar disorder treatments generally include one or more of the following: mood stabilizers; antidepressants; antipsychotics; and electroconvulsive therapy (ECT). There are largely three main types of drugs used for the treatment of bipolar disorder. These are lithium, anticonvulsants (e.g., Depakote or other valproate products) and atypical neuroleptics (e.g., risperidone, olanzapine, ziprasidone, aripiprazole, and quetiapine). These treatments may be used to treat both adults and children. Children, however, remain difficult to treat with many children not responding well to or refractory to treatments designed for adult-like symptoms or manifestation of the disorder.

The side effects of these drugs are particularly troublesome, and can be worse in children. These include the following. Atypical neuroleptics (except aripiprazole) are associated with marked weight gain in many children. The dangers of this weight gain include glucose problems that may include the onset of diabetes and increased blood lipids that may worsen heart and stroke problems later in life. In addition, these drugs can cause an illness called tardive dyskinesia, which involves repetitive movements including unsightly, repeated movements of the tongue in and out of the mouth or cheek. Depakote may also be associated with increased weight and possibly with a disease called polycystic ovarian syndrome (POS). In some cases POS is associated with infertility later in life. Lithium has been on the market the longest and is the only medication that has been shown to be effective against future episodes of mania, depression and completed suicides. Some people who take lithium over a long time will need a thyroid supplement and in rare cases may develop serious kidney disease. These side effects need to be weighed against the dangers of the manic-depressive illness itself, which can rob children of their childhood.

Thus, it is clear that improved methods and compositions for the diagnosis and treatment of bipolar disorder, including childhood-onset bipolar disorder, are needed.

Throughout this description, including the foregoing description of related art, any and all publicly available documents described herein, including any and all U.S. patents, are specifically incorporated by reference herein in their entirety. The foregoing description of related art is not intended in any way as an admission that any of the documents described therein, including pending United States patent applications, are prior art to embodiments of the present disclosure. Moreover, the description herein of any disadvantages associated with the described products and methods is not intended to limit the disclosed embodiments. Indeed, embodiments of the present disclosure may include certain features of the described products and methods without suffering from their described disadvantages.

### SUMMARY OF THE DISCLOSURE

The present invention provides for compositions and methods for treating bipolar disorder. According to some embodiments, compositions and methods are provided for treating mania associated with bipolar disorder. According to some embodiments, compositions and methods are provided for treating childhood-onset bipolar disorder. In general, a human in need of such treatment is one who has or expresses symptoms (e.g., mania) associated with bipolar disorder.

In some embodiments, the subject is a child under the age of 18 years (e.g., under the age of 17, 16, 15, 14, 13, 12, 11, 10, 9, 8, 7, 6, 5, 4, 3, or 2). In some embodiments, the subject is a child at least 18 months old. In some embodiments, the subject is a child between the stages of birth and puberty. In some embodiments, the subject is an adolescent child. Examples of age ranges of the subject according to some embodiments include between the ages of 2 to 18, between the ages of 2 to 15, between the ages of 2 to 12, between the ages of 2 to 10, between the ages of 2 to 8, between the ages of 2 to 6, between the ages of 2 to 4, between the ages of 4 to 15, between the ages of 4 to 12, between the ages of 4 to 10, between the ages of 4 to 8, between the ages of 4 to 6, between the ages of 6 to 18, between the ages of 6 to 15, between the ages of 6 to 12, between the ages of 6 to 10, between the ages of 6 to 8, between the ages of 8 to 18, between the ages of 8 to 15, between the ages of 8 to 12, and between the ages of 8 to 10.

According to some embodiments, there is provided methods for treating symptoms associated with bipolar disorder comprising administering to a subject, such as a human, in need of such treatment, a dose of ketamine sufficient to reduce or eliminate the symptoms associated with bipolar disorder.

According to some embodiments, there is provided methods for treating mania associated with bipolar disorder comprising administering to a subject, such as a human, in need of such treatment, a dose of ketamine sufficient to reduce or eliminate mania associated with bipolar disorder. In some embodiments, the subject is a child under the age of 18 years (e.g., under the age of 17, 16, 15, 14, 13, 12, 11, 10, 9, 8, 7, 6, 5, 4, 3, or 2). In some embodiments, the subject is a child at least 18 months old. In some embodiments, the subject is a child between the stages of birth and puberty. In some embodiments, the subject is an adolescent child.

According to some embodiments, there is provided methods for treating childhood-onset bipolar disorder comprising administering to a subject, such as a human, in need of such treatment, a dose of ketamine sufficient to reduce or eliminate the symptoms of childhood-onset bipolar disorder. In some embodiments, the treatment may begin when the subject is a child under the age of 18 years (e.g., under the age of 17, 16, 15, 14, 13, 12, 11, 10, 9, 8, 7, 6, 5, 4, 3, or 2). In some embodiments, the treatment may begin when the subject is a child at least 18 months old. In some embodiments, the treatment may begin when the subject is a child between the stages of birth and puberty. In some embodiments, the treatment may begin when the subject is an adolescent child. In some embodiments, the treatment may begin when the subject is an adult with childhood-onset or early-onset bipolar disorder.

In some embodiments, ketamine is administered intranasally. Thus, according to some embodiments, methods are provided for treating childhood-onset bipolar disorder in a subject comprising intranasally administering a therapeutic composition comprising ketamine to a subject in need thereof. According to some embodiments, methods are provided for treating mania in a subject comprising intranasally administering a therapeutic composition comprising ketamine to a subject in need thereof. In some embodiments, the subject is a child under the age of 18 years (e.g., under the age of 17, 16, 15, 14, 13, 12, 11, 10, 9, 8, 7, 6, 5, 4, 3, or 2). In some embodiments, the subject is a child at least 18 months old. In some embodiments, the subject is a child between the stages of birth and puberty. In some embodiments, the subject is an adolescent child.

According to some embodiments, the ketamine is in a pharmaceutically acceptable carrier and is administered at a dose of between about 0.1 mg/kg per day to about 3.0 mg/kg/day. In some embodiments, the doses of ketamine that are administered according to the method of the present invention are suitably low levels of ketamine which are below those which produce psychotomimetic side effects or are used for pain relief or control.

It is to be understood that both the foregoing general description and the following detailed description are exemplary, but not restrictive, of the invention.

### DETAILED DESCRIPTION OF THE INVENTION

The present invention is directed to a method for treating bipolar disorder, including childhood-onset bipolar disorder. In some embodiments, methods are provided for treating symptoms associated with bipolar disorder. The methods of the present invention comprise administering to a subject at least one dose of ketamine sufficient to alleviate symptoms associated with bipolar disorder. In some embodiments, the subject is a human in need of such treatment. A human in need of such treatment is one who has or expresses symptoms associated with bipolar disorder.

Ketamine ((2-(2-chlorophenyl)-2-(methylamino)-cyclohexanone) is a general anesthetic used by anesthesiologists, veterinarians, and researchers. Nasal administration of ketamine and midazolam to achieve sedation for ophthalmic surgery, and to induce anesthesia prior to elective surgery in healthy children has been reported. Usually, ketamine is administered intramuscularly (i.m.) or intravenously (i.v.) to induce anesthesia. However, intranasal compositions of ketamine are available from e.g., Javelin Pharmaceuticals. See e.g., U.S. Publication No. 2007/0287753 and U.S. Pat. No. 5,543,434, incorporated herein by reference in their entireties.

According to the present invention, the term “ketamine” refers to ketamine [(2-o-chlorophenyl)-2-(methylamino)-cyclohexanone], metabolites of ketamine, such as norketamine, pharmaceutically acceptable salts thereof, such as ketamine tannate, ketamine maleate, ketamine hydrochloride, etc., and biologically equivalent derivatives and analogs thereof, such as ketamine aspartate and ketamine succinate. Other names for ketamine include KETAJECT, KETALAR, KETANEST, KETASET, KETALAR, CALYPOS, and FELDEROSS. Branded names include Ereska™ (intranasal ketamine). Also included within the scope of the term “ketamine,” as one of ordinary skill would presume, are isomers and enantiomers thereof that demonstrate bipolar disorder-symptom treating properties.

According to some embodiments, there is provided methods for treating bipolar disorder in a subject comprising administering nasally a therapeutic composition comprising ketamine to a subject in need thereof. According to some embodiments, there is provided methods for treating mania in a subject comprising administering nasally a therapeutic composition comprising ketamine to a subject in need thereof. In some embodiments, the ketamine composition is self-administered nasally on an outpatient basis.

According to some embodiments, there is provided methods for treating childhood-onset bipolar disorder in a subject comprising administering nasally a therapeutic composition comprising ketamine to a subject in need thereof. According to some embodiments, there is provided methods for treating childhood-onset mania in a subject comprising administering nasally a therapeutic composition comprising ketamine to a subject in need thereof. In such methods, the subject may be under the age of 18. In some embodiments, the ketamine composition is self-administered nasally on an outpatient basis.

Childhood-onset bipolar disorder may be detected using any method known in the art. One method is use of the Childhood Bipolar Questionnaire (CBQ) as described in Papolos et al., J. Affect. Disord. 99: 27-36 (2007), incorporated herein by reference in its entirety. In a prior concordance study, of childhood-onset bipolar disorder, a behavioral phenotype termed Fear of Harm (FOH) was found to have one of the strongest concordance coefficients between subjects and siblings and the widest contrast between concordance coefficients for subject/sibling versus subject/comparison pairs. (See Papolos et al. (2007)). The FOH phenotype has been associated with children with childhood-onset bipolar disorder with increased mania and depression and other indices which demonstrate increased severity of illness. This association is described in greater detail in the Example. Thus, in some preferred embodiments, subjects are tested for childhood-onset bipolar disorder and for FOH. In other preferred embodiments, subjects are chosen for treatment because they have been diagnosed with childhood-onset bipolar disorder and art FOH positive.

The ketamine composition may be administered intranasally as a single dose, such as a single daily dose. The ketamine composition may be administered intranasally in multiple doses, such as multiple doses (e.g., 2, 3, 4, 5, 6, 7, 8, 9, 10, 15, 20, 25, and 50) doses of ketamine over a course of a day, days, weeks, or months. For example, the ketamine composition may be administered intranasally twice daily or four times weekly.

The ketamine composition may be administered intranasally with a dose of ketamine of approximately 0.01 to approximately 1 mg/kg of body weight. The ketamine composition may be administered intranasally with a dose of ketamine is approximately 0.05 to approximately 0.7 mg/kg of body weight.

The ketamine composition further comprises a dispersant. The dispersant may be a surfactant.

In some embodiments, the ketamine composition may be a dry powder aerosol formulation in which the ketamine is present as a finely divided powder. The dry powder aerosol formulation may further comprise a bulking agent. Without limitation, the bulking agent may be one or more of the following: lactose, sorbitol, sucrose, and mannitol.

In some embodiments, the ketamine composition may be a liquid aerosol formulation. The liquid aerosol formulation may further comprise a pharmaceutically acceptable diluent. Without limitation, the diluent may be one or more of the following: sterile water, saline, buffered saline, and dextrose solution.

According to the present invention, the terms “therapeutically effective dose”, “pharmaceutically effective dose”, or “effective dose” of ketamine is a dose that is generally effective in alleviating, reducing, noticeably reducing, or eliminating, symptoms associated with bipolar disorder or mania.

In some embodiments, the ketamine is introduced into the subject in the aerosol form in an amount between about 0.01 mg per kg body weight to about 1 mg per kg body weight. In a specific embodiment, the dosage is administered as needed. One of ordinary skill in the art can readily determine a volume or weight of aerosol corresponding to this dosage based on the concentration of ketamine in an aerosol formulation of the invention.

The dose of ketamine that is administered generally will depend on the size of the subject being treated. According to some embodiments, the ketamine is in a pharmaceutically acceptable carrier and is administered at a dose of between about 0.1 mg/kg per day to about 3.0 mg/kg/day. The dose of ketamine may be from approximately 0.01 to approximately 1 mg/kg of body weight. In some embodiments, the dose of ketamine is approximately 0.05 to approximately 0.7 mg/kg of body weight. In other embodiments, the total dose of ketamine per nasal administration ranges from about 1 to about 250 mg. A dose of any integer between these two numbers is contemplated. Thus, for example, intranasal formulations respectively containing total intranasal doses of 1 mg, 2 mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, and 250 mg are specifically contemplated.

In some embodiments, the amount of ketamine administered to a patient suffering from bipolar disorder is from about 10% to about 20% of the amount used to induce anesthesia.

As bipolar disorder or mania is a chronic illness requiring maintenance treatment, it is expected that chronic administration of the intranasal formulation may be employed as necessary, ranging from daily to weekly, depending on response. For example, should the 50 mg intranasal dosage prove to be inadequate to treat bipolar disorder or mania effectively, increasing doses, e.g., approximately 100 mg, approximately 150 mg, approximately 200 mg, approximately 250 mg total ketamine may be administered intranasally.

The doses of ketamine used may be suitably modified to take into account the ketamine bioavailability so that the serum level of ketamine is less than, or on the order of 50 ng/ml.

Doses of ketamine far lower than those used in the treatment of other disorders are beneficial for subjects suffering from bipolar disorder or mania, in order to minimize the psychotomimetic side effects commonly associated with the use of the higher doses of ketamine. Also, it is suitable that the dose of ketamine administered is less than the dose generally administered for alleviation of pain.

The mild adverse effects of ketamine, e.g., dysphoria and/or hallucinations, sometimes called “ketamine dreams,” can occur upon administration of a dose of greater than 50 mg of ketamine, and usually require doses greater than 100 mg of ketamine of total dose intranasally. One advantage of the present invention is that nasal delivery of ketamine allows for control of the dose to a level effective for analgesia, but below the level that results in such dreams.

For purposes of treatment in accordance with the present invention, it is suitable that the dose of ketamine is administered intranasally. The term “nasal administration” in all its grammatical forms refers to administration of a drug through the nasal mucous membrane to the bloodstream for systemic delivery of the drug. The advantages of nasal administration for drug delivery are that it does not require injection using a syringe and needle, it avoids necrosis that can accompany i.m. administration of drugs, and trans-mucosal administration of a drug is highly amenable to self administration.

Ketamine will preferably be prepared in a formulation or pharmaceutical composition appropriate for nasal administration. In a further embodiment, ketamine can be formulated with a mucosal penetration enhancer to facilitate delivery of the drug. The formulation can also be prepared with pH optimized for solubility, drug stability, absorption through nasal mucosa, and other considerations.

The ketamine may be formulated with a “mucosal penetration enhancer,” i.e., a reagent that increases the rate or facility of transmucosal penetration of ketamine, such as but not limited to, a bile salt, fatty acid, surfactant or alcohol. In specific embodiments, the permeation enhancer can be sodium cholate, sodium dodecyl sulphate, sodium deoxycholate, taurodeoxycholate, sodium glycocholate, dimethylsulfoxide or ethanol.

The present invention contemplates formulations comprising ketamine for use in a wide variety of devices that are designed for the delivery of pharmaceutical compositions and therapeutic formulations to the respiratory tract, preferably the nasal passages. Any device known in the art for the nasal delivery of therapeutic agents may be applied for use in the methods of administering ketamine to the nasal passages.

A preferred route of administration of the present invention is in an aerosol spray for nasal inhalation. With regard to construction of the delivery device, any form of aerosolization known in the art, including but not limited to spray bottles, nebulization, atomization or pump aerosolization of a liquid formulation, and aerosolization of a dry powder formulation, can be used in the practice of the invention. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Ketamine, combined with a dispersing agent, or dispersant, can be administered in an aerosol formulation as a dry powder or in a solution or suspension with a diluent. As used herein, the term “aerosol” refers to suspension in the air. In certain embodiments, the ketamine can also be combined with a preservative. Optionally, this preservative is benzalkonium quaternary ammonium preservative. In some embodiments, the preservative is benzalkonium chloride. In particular, aerosol refers to the particlization or atomization of a formulation of the invention and its suspension in the air. According to the present invention, an aerosol formulation is a formulation comprising ketamine for nasal inhalation or pulmonary administration.

As used herein, the term “inhaler” refers both to devices for nasal and pulmonary administration of a drug, e.g., in solution, powder and the like. For example, a the term “inhaler” is intended to encompass a propellant driven inhaler, such as is used for to administer antihistamine for acute asthma attacks, and plastic spray bottles, such as are used to administer decongestants.

In some embodiments, the device for aerosolization is a metered dose inhaler. A metered dose inhaler provides a specific dosage when administered, rather than a variable dose depending on administration. Such a metered dose inhaler can be used with either a liquid or a dry powder aerosol formulation. Metered dose inhalers are well known in the art.

As used herein, the term “dispersant” refers to an agent that assists aerosolization of the ketamine or absorption of the ketamine in mucosal tissue, or both. In a specific aspect, the dispersant can be a mucosal penetration enhancer. Preferably, the dispersant is pharmaceutically acceptable. As used herein, the term “pharmaceutically acceptable” means approved by a regulatory agency of the Federal or a state government or listed in the U.S. Pharmacopeia or other generally recognized pharmacopeia for use in animals, and more particularly in humans.

Suitable dispersing agents are well known in the art, and include but are not limited to surfactants and the like. Such surfactants are generally used in the art to reduce surface induce aggregation of ketamine caused by atomization of the solution forming the liquid aerosol and may be used in the methods and devices of the present invention. Examples of such surfactants include, but are not limited to, surfactants such as polyoxyethylene fatty acid esters and alcohols, and polyoxyethylene sorbitan fatty acid esters. Amounts of surfactants used will vary, being generally within the range or 0.001 and 4% by weight of the formulation. Suitable surfactants are well known in the art, and can be selected on the basis of desired properties, depending on the specific formulation, concentration of ketamine, diluent (in a liquid formulation) or form of powder (in a dry powder formulation), etc.

The liquid aerosol formulations contain ketamine and a dispersing agent in a physiologically acceptable diluent. The dry powder aerosol formulations of the present invention consist of a finely divided solid form of ketamine and a dispersing agent. With either the liquid or dry powder aerosol formulation, the formulation must be aerosolized. That is, it must be broken down into liquid or solid particles in order to ensure that the aerosolized dose actually reaches the mucous membranes of the nasal passages or the lung. The term “aerosol particle” is used herein to describe the liquid or solid particle suitable for nasal or pulmonary administration, i.e., that will reach the mucous membranes. Other considerations, such as construction of the delivery device, additional components in the formulation, and particle characteristics are important. These aspects of nasal or pulmonary administration of a drug are well known in the art, and manipulation of formulations, aerosolization means and construction of a delivery device require at most routine experimentation by one of ordinary skill in the art.

In some embodiments, the mass median dynamic diameter will be 5 micrometers or less in order to ensure that the drug particles reach the lung alveoli.

For nasal administration, a useful device is a small, hard bottle to which a metered dose sprayer is attached. In one embodiment, the metered dose is delivered by drawing the ketamine solution into a chamber of defined volume, which chamber has an aperture dimensioned to aerosolize and aerosol formulation by forming a spray when a liquid in the chamber is compressed. The chamber is compressed to administer the ketamine. In a specific embodiment, the chamber is a piston arrangement. Such devices are commercially available.

Alternatively, a plastic squeeze bottle with an aperture or opening dimensioned to aerosolize an aerosol formulation by forming a spray when squeezed. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

Often, the aerosolization of a liquid or a dry powder formulation for inhalation into the lung will require a propellent. The propellent may be any propellant generally used in the art. Specific nonlimiting examples of such useful propellants are a chloroflourocarbon, a hydrofluorocarbon, a hydrochlorofluorocarbon, or a hydrocarbon, including trifluoromethane, dichlorodifluoromethane, dichlorotetrafluoroethanol, and 1,1,1,2-tetrafluoroethane, or combinations thereof.

The present invention provides liquid aerosol formulations and dosage forms for use in treating subjects suffering from bipolar disorder, including childhood-onset bipolar disorder. In general such dosage forms contain ketamine in a pharmaceutically acceptable diluent. Pharmaceutically acceptable diluents in such liquid aerosol formulations include but are not limited to sterile water, saline, buffered saline, dextrose solution, and the like. In a specific embodiment, a diluent that may be used in the present invention or the pharmaceutical formulation of the present invention is phosphate buffered saline or a buffered saline solution generally between the pH 7.0-8.0 range, or water.

The liquid aerosol formulation also may optionally include pharmaceutically acceptable carriers, diluents, solubilizing or emulsifying agents, surfactants and excipients.

The formulation may include a carrier. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable plasma half life for clearance. Such macromolecules include but are not limited to Soya lecithin, oleic acid and sorbitan trioleate, with sorbitan trioleate preferred.

The formulations of the present embodiment may also include other agents useful for pH maintenance, solution stabilization, or for the regulation of osmotic pressure. Examples of the agents include but are not limited to salts, such as sodium chloride, or potassium chloride, and carbohydrates, such as glucose, galactose or mannose, and the like.

The present invention further contemplates liquid aerosol formulations comprising ketamine and another therapeutically effective drug, such as a described in further detail below.

Where a pharmaceutically acceptable preservative is to be included in the formulations of the invention, the preservative is selected from the group consisting of phenol, m-cresol, benzalkonium chloride, chloroethanol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, or mixtures thereof. Each one of these specific preservatives constitutes an alternative embodiment of the invention. In a preferred embodiment of the invention the preservative is benzyl alcohol, a phenol, or m-cresol.

In a further embodiment of the invention the preservative is present in a concentration from about 0.1 mg/ml to about 50 mg/ml, more preferably in a concentration from about 0.1 mg/ml to about 25 mg/ml, and most preferably in a concentration from about 0.1 mg/ml to about 10 mg/ml.

It is also contemplated that the present aerosol formulation can be prepared as a dry powder formulation comprising a finely divided powder form of ketamine and a dispersant. For example, the dry powder formulation can comprise a finely divided dry powder containing ketamine, a dispersing agent and also a bulking agent. In a further embodiment, the formulation may further comprise a bulking/stabilizing agent. Bulking/stabilizing agents include, but are not limited to, the following: sucrose, fructose, dextrose, maltose, glucose, dextran, mannitol, sorbitol, inositol, galactitol, xylitol, lactose, trehalose or human serum albumin. Each one of these specific stabilizers constitutes an alternative embodiment of the invention. In a preferred embodiment of the invention the stabilizer is mannitol.

In a further embodiment, the bulking/stabilizing agent is present in a concentration from 0.1 mg/ml to 50 mg/ml. In a further embodiment of the invention the bulking/stabilizing agent is present in a concentration from 0.1 mg/ml to 5 mg/ml. In a further embodiment of the invention the bulking/stabilizing agent is present in a concentration from 5 mg/ml to 10 mg/ml. In a further embodiment of the invention the bulking/stabilizing agent is present in a concentration from 10 mg/ml to 20 mg/ml. In a further embodiment of the invention the bulking/stabilizing agent is present in a concentration from 20 mg/ml to 30 mg/ml. In a further embodiment of the invention the bulking/stabilizing agent is present in a concentration from 30 mg/ml to 50 mg/ml.

In a further embodiment of the invention the formulation of the invention may further comprise an antioxidant.

Buffers may be added to maintain the pH of the formulation to between about 3 to about 8 (e.g., about 3, about 3.5, about 4, about 4.5, about 5, about 5.5, about 6, about 6.5, about 6.8, about 7, about 7.2, about 7.3, about 7.4, about 7.5, about 7.6). According to some embodiments, the pH of the formulation may be between about 3 and about 6, between about 3 and about 5.5, between about 3 and about 5.2, between about 3 and about 4.5, between about 3 and about 4, between about 4 and about 5.5, between about 4.5 and about 5.5, between about 4 and about 6, between about 4 and about 7, between about 3 and about 7, between about 4 and about 7.5, between about 5 and about 7.5, between about 5 and about 6.5, between about 5 and about 8, between about 6 and about 8, or between about 6.5 and about 7.5.

Any pharmaceutically acceptable buffer may be used in the present formulations/compositions. Preferably, the buffer is present in a concentration of from about 1 mM to about 100 mM (e.g., about 5 mM, about 10 mM, about 15 mM, about 20 mM, about 25 mM, about 30 mM, about 35 mM, about 40 mM, about 50 mM, about 55 mM, about 60 mM, about 65 mM, about 70 mM, about 75 mM, about 80 mM, about 85 mM, about 90 mM, or about 95 mM).

The buffer to be included in the formulations may include, but is not limited to, the following: acetate (e.g., sodium acetate), sodium carbonate, citrate (e.g., sodium citrate), tartrate, glycylglycine, histidine, glycine, lysine, arginin, sodium dihydrogen phosphate, disodium hydrogen phosphate, sodium phosphate, and tris(hydroxymethyl)-aminomethan, or mixtures thereof. Each one of these specific buffers constitutes an alternative embodiment of the invention. In a preferred embodiment of the invention the buffer is glycylglycine, sodium dihydrogen phosphate, disodium hydrogen phosphate, sodium phosphate or mixtures thereof.

Sterility or adequate antimicrobial preservation may be provided as part of the present formulations. It is preferred that they be free of pathogenic organisms. A benefit of a sterile liquid suspension is that it reduces the possibility of introducing contaminants into the individual when the suspension formulation is administered to the nasal passages, thereby reducing the chance of an opportunistic infection. Processes which may be considered for achieving sterility may include any appropriate sterilization steps known in the art. In one embodiment, the ketamine is produced under sterile conditions, the micronization is performed in a sterile environment, and the mixing and packaging is conducted under sterile conditions. In alternative embodiment, the formulations of the present invention may be sterile filtered and filled in vials, including unit dose vials providing sterile unit dose formulations which are used in a nasal spray device for example. Each unit dose vial may be sterile and is suitably administered without contaminating other vials or the next dose. In one alternative embodiment, one or more ingredients in the present formulation may be sterilized by steam, gamma radiation or prepared using or mixing sterile steroidal powder and other sterile ingredients where appropriate. Also, the formulations may be prepared and handled under sterile conditions, or may be sterilized before or after packaging.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only not intended to be limiting. Other features and advantages of the invention will be apparent from the following detailed description and claims.

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth.

### EXAMPLE

### Linking of Fear of Harm (FOH) Phenotype with Childhood-Onset Bipolar Disorder

Methods

A sample was comprised of children with community diagnoses of bipolar disorder or at risk for the illness based on enriched family history with multiple first degree relatives diagnosed with bipolar disorder (BPD) (N=5335). Included were all subjects who had N40 positively endorsed Childhood Bipolar Questionnaire (CBQ) symptom items at frequencies of very often, almost always, and always. This group was divided randomly into two groups, the exploratory group (N=2668) and the study group (N=2666). The exploratory group was used for exploratory data analysis and the development of hypotheses. A study group was used to test these hypotheses using a new and uncontaminated set of data. All results reported here are derived from the latter group. In subsequent analyses, a subset of the study group sample was examined for differences in age of onset of first psychiatric symptoms, course of illness and measures of symptom severity. These groups were compared using the chi-square procedure for categorical data and the Analysis of Variance (ANOVA) with Scheffe pair wise tests for continuous variables. The Child Bipolar Questionnaire V.2.0, the Yale-Brown Obsessive Compulsive Scale (YBOCS) and the Overt Aggression Scale (OAS) were the principal instruments used to obtain diagnostic information for this study.

To assess the relationship between membership in an FOH group and symptoms of mood dysregulation and psychiatric disorders, the CBQ was administered to all subjects (N=1726). The CBQ is a 65 item, self-administered, parent report measure originally developed to establish initial eligibility for clinical and genetic studies of PBD (Papolos et al., J. Affect. Disord. 95: 149-158 (2006), incorporated herein by reference in its entirety). It was constructed based on the model proposed by Depue et al., J. Abnorm. Psychol. 90: 381-437 (1981), incorporated herein by reference in its entirety, who, with the development and validation of the General Behavior Inventory (GBI), derived a dimensional approach for the definition of BPD in adults. Behaviors and symptoms are measured on 1-4 Likert scale. A rapid screening instrument with a Core Index subscale of key symptom dimensions frequently reported in PBD, the CBQ includes scoring algorithms for DSM-IV BD, with and without attention deficit/hyperactivity disorder (ADHD). Test/retest data showed excellent reliability for both the CBQ total score (r=0.82) and the Core Index subscale (r=0.86). CBQ screening algorithms were performed with a specificity of 97% and a sensitivity of 76% in classifying subjects with Kiddie Schedule for Affective Disorders and Schizophrenia (K-SADS P/L) diagnosis of BD vs. no BD (Papolos et al., 2006). The Core Index subscale had excellent agreement with K-SADS P/L diagnosis (k=0.84) in classifying BD, ADHD-only, and no diagnosis and demonstrated 100% sensitivity and 86% specificity in classifying BD vs. no BD. Consistent with a previous examination of the FOH symptom dimension (Papolos et al., J. Affect. Disord. 89: 99-105 (2005a), incorporated herein by reference in its entirety), a Yale-Brown Obsessive Compulsive Scale (YBOCS) measure was used that consisted of a count of six aggressive obsessions rated by the parent as occurring at a frequency of “often” or “very often” or “almost constantly”: fear might harm self; fear might harm others; fear harm might come to self; fear harm will come to others (may be because of something child did or did not do); fear will act on unwanted impulses (e.g., to stab a family member); and fear will be responsible for something else terrible happening (e.g., fire, burglary, flood). The FOH index was calculated by summing six YBOCS items that had scored greater or equal to 3 and two items from OAS that had scored greater or equal to 2. The items from the OAS are: mutilates self, causes deep cuts, bites that bleed internal injury, fracture, loss of consciousness, loss of teeth and attacks others, causing severe physical injury.

Consistent with a previous examination of the FOH symptom dimension (Papolos et al., 2005a and Papolos et al. J. Affect. Disord. 86: 267-275 (2005b), incorporated herein by reference in its entirety) YBOCS items that had scored greater or equal to 3 and two items from OAS that scored greater or equal to 2 defined the phenotype. A principal component factor analysis with Varimax rotation was used to determine what other traits are associated with the FOH trait by examining the independent factors derived from the CBQ. To determine the nature and extent to which each of these factors were associated with the FOH trait, a total score for each factor was calculated by summing all items for each factor and the factors were named based on item content. Cronbach's alpha was also calculated per factor. These factors were used in a multiple regression model to predict the Fear of Harm Index using a stepwise method. Some questions were not applicable to all subjects, resulting in different sample size per variable. The SPSS version 15 was used for all these analyses.

Results

Of the 2666 subjects, 1729 were found to have FOH data. When the distribution of the FOH index in this sample was examined, it was found that a full third of the group had no FOH (X²=169.14, df=1, pb.001). The total group of 1729 children was, therefore, divided into three groups. A group with no FOH symptoms (NoFOH), values of 0 positively endorsed items (NoFOH: 0±0, N=621, 36%), and subjects with values from 1 through 7 (LowFOH: 4.5±2N=555, 32%) were designated as the low FOH group. The high FOH group included subjects with values greater than or equal to seven (HighFOH: 14.1±5, N=553, 32%).

Although there were no significant differences between rates of males and females on the Fear of Harm Index (female: 5.7±6, male: 6.2±7, f=2.1, df=1.1640, p=0.148), there were significantly more male subjects in the LowFOH group (NoFOH: 35%, LowFOH: 45%, and HighFOH: 34%, X²=6.41, df=2, p=0.041). There was no significant age difference among groups (NoFOH=10.0±4, N=585; LowFOH=10.2±4, N=528; HighFOH=10.4±4; f=1.7; df=2.1636; p=0.182). However, there were significantly more ADHD subjects in the NoFOH group compared to HighFOH (NoFOH=19%, Low-FOH=16%, HighFOH=11%, X²=7.9, df=1, p=0.005).

Despite the fact that the three groups did not differ on the number of subjects diagnosed with bipolar disorder (NoFOH=83%, LowFOH=86%, HighFOH=86%, X²=1.13, df=2, p=0.57), or major depressive disorder (NoFOH=4%, LowFOH=2%, HighFOH=2%, X²=2.69, df=2, p=0.26), using CBQ item scores we found that there was a significantly greater frequency of manic (NoFOH=5.0±2, LowFOH=5.7±1, HighFOH=5.6±2; f=79.43; df=2.1726; pb.0001) and depressive symptoms (NoFOH=3.9±2, LowFOH=4.6±2, HighFOH=4.9±2; f=60.53; df=2.1726; pb.0001) in the high FOH group when compared to the low or no FOH groups. Pairwise tests indicate that all groups are significantly different from each other on these variables. These differences are also evident when the dimensions were dichotomized (Table 1). The HighFOH group has a significantly greater number of subjects with five or more manic/hypomanic symptoms, 91%, compared to the LowFOH group of 83% and NoFOH group of 69% of subjects (X²=93.8, df=2, pb.000). All pair wise comparisons were also significant. The differences persisted when analyzed for depressive symptoms; 84% of the HighFOH group exhibited four or more symptoms of depression in comparison to 78% of the LowFOH and 62% of NoFOH groups (X²=76.4, df=2, pb.0001). All pair wise comparisons were also significant. Similar results were found when groups were compared separately for male and female subjects (Table 1).

TABLE 1
Group differences on symptoms of mania and depression
mania and depression symptoms (N = 1729).

| | NoFOH | LowFOH | HighFOH | X²* |
|---|---|---|---|---|
| Manic symptoms greater or | 69% (426) | 83% (459) | 91% (502) | 93.8 |
| equal to 5 | | | | |
| Depressive symptoms greater | 62% (387) | 78% (431) | 84% (464) | 76.4 |
| or equal to 4 | | | | |

*p < .001.

Course of illness data were available for 967 children. Within this subgroup the same criteria was applied for FOH status. Similar to the larger pool of children, this smaller group contained about a third of children who endorsed 0 items of FOH (N=334, 35%), a third endorsed 1 through 7 items (N=322, 33%) and a third endorsed more than 7 items (N=311, 32%). The similarity of the distribution of FOH in each group raises one's confidence that this smaller subset of children is a representative sample of the larger sample.

The three groups endorsed CBQ items significantly differently from each other (f=137.69; df=2.981; pb.001). The NoFOH group positively endorsed 37.9±11 items, LowFOH 45.8±8 items and the HighFOH group positively endorsed 49.6±8 items. These differences were similar to the larger group. The groups were not significantly different in age (NoFOH=9.7±4, LowFOH=9.9±4, HighFOH=10.3±4; f=2.10; df=2.896; p=0.122). The groups had a similar distribution of male subjects (NoFOH: 33%, LowFOH: 30%, and HighFOH: 36%, X2=5.11, df=2, p=0.077).

These groups had a similar age of onset of first reported psychiatric symptoms, age of initial psychiatric evaluation, age of initial diagnosis and age at first psychiatric hospitalization. However, they were significantly different on the number of hospitalizations (Table 2).

TABLE 2
Course of illness (N = 967).

| | NoFOH | LowFOH | HighFOH | f | p < .01 |
|---|---|---|---|---|---|
| Age of 1st symptoms (years) | 2.7 ± 2 (N = 334) | 2.6 ± 3 (N = 322) | 2.5 ± 2 (N = 311) | 1.12 | .326 |
| Age of initial psychiatric evaluation (years) | 6.0 ± 3 (N = 316) | 6.0 ± 3 (N = 312) | 6.0 ± 3 (N = 300) | .067 | .963 |
| Age of initial diagnosis (years) | 6.3 ± 3 (N = 306) | 6.5 ± 5 (N = 313) | 6.3 ± 4 (N = 302) | .365 | .694 |
| Age of 1st psychiatric hospitalization (years) | 9.7 ± 4 (N = 78) | 9.6 ± 4 (N = 114) | 9.4 ± 4 (N = 164) | .337 | .713 |
| Number of hospitalizations | 1.5 ± 1 (N = 86) | 1.8 ± 2 (N = 118) | 2.4 ± 2 (N = 172) | 6.31 | .002* |

*Significant pair wise comparisons based on Scheffe formula: NoFOH vs. LowFOH (p = .005) and vs. HighFOH (p = .044).

The NoFOH group has a significantly fewer number of hospitalization than the other two groups.

On measures of severity of illness presented in Table 3, there were significant differences found among the FOH groups on the severity of illness variables; Ever Hospitalized, Held Back a Grade, and Suspended from School.

TABLE 3
Group differences on measures of severity of illness.

| | Yes | NoFOH (%) | LowFOH (%) | HighFOH (%) | X² | p value |
|---|---|---|---|---|---|---|
| Ever hospitalized (N = 984) | 352 | 22 | 34 | 52 | 63.7 | .001 |
| Home schooled (N = 984) | 40 | 5.4 | 4.3 | 2.2 | 4.7 | .094 |
| Held back a grade (N = 880) | 171 | 15 | 20 | 24 | 8.5 | .014 |
| Ever suspended from school (N = 905) | 366 | 36 | 38 | 48 | 9.9 | .007 |
| Involved with the juvenile justice system (N = 984) | 110 | 91 | 89 | 86 | 5.1 | .079 |

However, the groups were not significantly different on home schooling and their involvement with the juvenile justice system. All groups were significantly different from each other on ever hospitalized with HighFOH has the largest percentage of subjects (52%). Significantly more subjects from the HighFOH group were also held back a grade compared to NoFOH (X²=8.49, df=1, p=0.004) and significantly more subjects from HighFOH and LowFOH were suspended from school than NoFOH (X²=8.48, df=1, p=0.004; X²=6.24, df=1, p=0.012). There was a strong trend between held back a grade and suspended from school. 47% of subjects who were held back a grade were also suspended from school (X²=2.75, df=1, p=0.098). 14% of subjects from HighFOH groups were suspended from school and held back a grade compared to 7% from NoFOH (X²=7.39, df=1, p=0.007) and 6% subjects from LowFOH groups (X²=11.30, df=1, p=0.001).

Using all of the children in the study, a principal component factor analysis was used to identify a set of independent dimensions associated with the FOH trait of children (N=1729; NoFOH=621, LowFOH=555, High-FOH=553). The factor analysis yielded thirteen factors with eigen values greater than 1.0 that explained a total of 61% of variance. By combining 3 of the factors with the lowest Cronbach's alpha with other factors to which they also contributed, the 13 factors were reduced to 10. These ten factors their CBQ items, eigenvalues, percentage of variance and the Cronbach's alphas are listed in Table 4.

TABLE 4

| Factor | CBQ items | Eigenvalues | % Variance | α |
|---|---|---|---|---|
| Factor 1: Territorial | 46) is willful and refuses to be subordinated by others | 16.56 | 25.5 | .91 |
| Aggression | 47) argues with adults | | | |
| | 49) defies or refuses to comply with rules | | | |
| | 51) is easily angered in response to limit setting | | | |
| | 48) is bossy towards others | | | |
| | 45) relentlessly pursues own needs and is demanding | | | |
| | of others | | | |
| | 50) blames others for his/her mistakes | | | |
| | 53) has protracted, explosive temper tantrums | | | |
| | 55) displays aggressive behavior towards others | | | |
| | 32) has irritable mood states | | | |
| | 52) lies to avoid consequences of his/her actions | | | |
| | 44) is intolerant of delays | | | |
| | 54) has difficulty maintaining friendships | | | |
| Factor 2: Attention/Executive | 17) has difficulty organizing tasks | 3.71 | 5.7 | .87 |
| function | 13) demonstrates inability to concentrate at school | | | |
| | 12) is easily distracted during repetitive chores and | | | |
| | lessons | | | |
| | 14) attempts to avoid homework assignments | | | |
| | 16) has poor handwriting | | | |
| | 11) is easily distracted by extraneous stimuli | | | |
| | 19) has difficulty estimating time | | | |
| | 15) able to focus intently on subjects of interest and | | | |
| | yet at times is easily distractible | | | |
| | 20) has auditory processing or short-term memory deficit | | | |
| | 18) has difficulty making transitions | | | |
| Factor 3: Mania | 25) has periods of high, frenetic energy and motor activation | 3.24 | 4.9 | .87 |
| | 28) has periods of excessive and rapid speech | | | |
| | 26) has many ideas at once | | | |
| | 33) has elated or silly, goofy, giddy mood states | | | |
| | 24) is easily excitable | | | |
| | 27) interrupts or intrudes on others | | | |
| | 04) is hyperactive and easily excited in the PM | | | |
| | 31) displays abrupt, rapid mood swings | | | |
| | 43) fidgets with hands or feet | | | |
| | 65) is very intuitive and/or very creative | | | |
| | 30) tells tall tales; embellishes or exaggerates | | | |
| | 29) has exaggerated ideas about self or abilities | | | |
| Factor 4: Harm to Self/Others | 59) makes clear threats of violence to others or self | 2.93 | | .83 |
| | 58) makes moderate threats to others or self | | | |
| | 60) has made clear threats of suicide | | | |
| | 57) curses viciously, uses foul language in anger | | | |
| | 56) has destroyed property intentionally | | | |
| | 61) is fascinated with gore, blood, or violent imagery | | | |
| Factor 5: Self-esteem | 41) feels easily criticized and/or rejected | 2.40 | | .84 |
| | 42) feels easily humiliated or shamed | | | |
| | 40) experiences periods of self doubt and poor self-esteem | | | |
| | 37) complains of being bored | | | |
| | 38) has periods of low energy and/or withdraws or | | | |
| | isolates self | | | |
| | 39) has decreased initiative | | | |
| Factor 6: Sleep | 06) has difficulty getting to sleep | 1.93 | | .74 |
| | 05) has difficulty settling at night | | | |
| | 07) sleeps fitfully and/or awakens in the middle of | | | |
| | the night | | | |
| | 03) has difficulty arising in the AM | | | |
| Factor 7: Sensory | 21) is extremely sensitive to textures of clothes, labels, | 1.78 | | .71 |
| | and tightness of fit of socks or shoes | | | |
| | 22) exhibits extreme sensitivity to sound and noise | | | |
| | 23) complains of body temperature extremes or feeling | | | |
| | hot despite neutral ambient temperature | | | |
| Factor 8: Hypersexuality | 34) displays precocious sexual curiosity | 1.50 | | .74 |
| | 35) exhibits inappropriate sexual behaviors, e.g. openly | | | |
| | touches self or others' private parts | | | |
| | 36) takes excessive risks | | | |
| Factor 9: Psychosis, | 09) wets bed | 1.23 | | .59 |
| Parasomnias, Sweet | 08) has night terrors and/or nightmares | | | |
| Cravings, | 63) hoards or avidly seeks to collect objects or food | | | |
| and Obsessions | 62) has acknowledged experiencing auditory and/or | | | |
| | visual hallucinations | | | |
| | 10) craves sweet-tasting foods | | | |
| | 64) has concern with dirt, germs, or contamination | | | |
| Factor 10: Anxiety | 01) displays excessive distress when separated from | 1.03 | | .66 |
| | family | | | |
| | 02) exhibits excessive anxiety or worry | | | |

α : Cronbach's alpha.

Descriptive information for each CBQ factor for the three FOH groups are presented in Table 5.

TABLE 5
Bipolar Child Questionnaire Factor Scores:
Mean Standard Deviations Across FOH Groups.

| | | | | Factor group |
|---|---|---|---|---|
| | NoFOH | LowFOH | HighFOH | mean |
| Territorial Aggression^{a} | 39.8 ± 9 | 43.5 ± 7 | 46.5 ± 5 | 43.1 ± 8 |
| Attention/Executive | 30.9 ± 7 | 33.1 ± 5 | 34.5 ± 5 | 32.8 ± 6 |
| function | | | | |
| Mania | 36.1 ± 7 | 39.0 ± 6 | 41.3 ± 5 | 38.7 ± 7 |
| Harm to Self/Others^{a} | 11.9 ± 4 | 14.5 ± 4 | 17.6 ± 4 | 14.5 ± 5 |
| Self-esteem^{a} | 17.3 ± 4 | 18.9 ± 4 | 20.1 ± 4 | 18.7 ± 4 |
| Sleep^{a} | 11.5 ± 3 | 12.5 ± 3 | 13.0 ± 3 | 12.3 ± 3 |
| Sensory | 7.4 ± 3 | 8.1 ± 3 | 8.8 ± 3 | 8.1 ± 3 |
| Hypersexuality | 5.7 ± 2 | 6.5 ± 3 | 7.3 ± 3 | 6.5 ± 3 |
| Psychosis/Parasomnias/Sweet | 11.5 ± 3 | 13.2 ± 3 | 14.6 ± 4 | 13.0 ± 4 |
| Cravings/Obsessions^{a} | | | | |
| Anxiety^{a} | 4.7 ± 2 | 5.5 ± 2 | 5.9 ± 2 | 5.3 ± 2 |

^{a}These are the significant factors that emerged with multiple regression analysis of the factor structure.

The mean number of CBQ items endorsed by the three FOH groups was significantly different from each other. The NoFOH group positively endorsed 37.9±11 items (out of 65 items), LowFOH 45.05±9 items and the HighFOH group positively endorsed 49.99±8 items (f=243.27; df=2.1726; pb.001). Subjects who scored either HighFOH or LowFOH were found to have more severe symptoms on all of these CBQ factors than children without the FOH trait.

We performed a logistic regression analysis of ten independent CBQ factors in order to determine which individual set of behavioral traits is the best predictor of FOH. A four factor model, (X²=1601, df=4, pb.001) including, Territorial Aggression, Harm to Self and Others, Selfesteem, and Psychosis/Parasomnias/Sweet Craving/Obsessions (PPSO) correctly predicted the FOH group with 96% accuracy.

Thus, subjects that were diagnosed with bipolar disorder using the CBQ who were positive for FOH had more hospitalizations, were more likely to be held back a grade, and more likely to be suspended from school than subjects with. Thus, subjects diagnosed with childhood-onset bipolar disorder with FOH may have more severe symptoms than subjects without FOH.

What is claimed is:

1. A method for treating childhood-onset bipolar disorder in a subject comprising intranasally administering a therapeutic composition comprising ketamine to a subject in need thereof.

2. The method of claim 1, wherein the subject is at least 18 months of age.

3. The method of claim 1, wherein the subject is a child between the stages of 18 months and puberty.

4. The method of claim 1, wherein the ketamine composition is self-administered nasally on an outpatient basis.

5. The method of claim 1, wherein said method comprises intranasally administering a single dose of ketamine.

6. The method of claim 1, wherein said method comprises intranasally administering multiple doses of ketamine.

7. The method of claim 1, wherein said method comprises intranasally administering a dose of ketamine of approximately 0.01 to approximately 1 mg/kg of body weight.

8. The method of claim 1, wherein the dose of ketamine is approximately 0.05 to approximately 0.7 mg/kg of body weight.

9. The method of claim 1, wherein the ketamine composition further comprises a dispersant.

10. The method of claim 1, wherein the ketamine composition is a dry powder aerosol formulation in which the ketamine is present as a finely divided powder.

11. The method of claim 1, wherein the ketamine composition is a liquid aerosol formulation.

12. A method for treating mania in a subject comprising intranasally administering a therapeutic composition comprising ketamine to a subject in need thereof.

13. The method of claim 12, wherein the subject is at least 18 months of age.

14. The method of claim 12, wherein the subject is a child between the stages of 18 months and puberty.

15. The method of claim 12, wherein the ketamine composition is self-administered nasally on an outpatient basis.

16. The method of claim 12, wherein said method comprises intranasally administering a single dose of ketamine.

17. The method of claim 12, wherein said method comprises intranasally administering multiple doses of ketamine.

18. The method of claim 12, wherein said method comprises intranasally administering a dose of ketamine of approximately 0.01 to approximately 1 mg/kg of body weight.

19. The method of claim 12, wherein the dose of ketamine is approximately 0.05 to approximately 0.7 mg/kg of body weight.

20. The method of claim 12, wherein the ketamine composition is a dry powder aerosol formulation in which the ketamine is present as a finely divided powder.

21. The method of claim 12, wherein the ketamine composition is a liquid aerosol formulation.

22. The method of claim 12, wherein the subject is an adult having childhood-onset bipolar disorder.
